(19) United States
(12) Patent Application Publication
(10) Pub. No.: US 2013/0144080 A1
(43) Pub. Date: Jun. 6, 2013

(54) PROCESS FOR OBTAINING A DIALKYL CARBONATE AND AN ALKYLENE GLYCOL

(71) Applicants: Andrea SCHMIDT, Ludwigshafen (DE); Michael Bock, Ruppertsberg (DE); Roderich Roettger, Langenhagen (DE); Robert Baumann, Mannheim (DE); Axel Franzke, Mannheim (DE)

(72) Inventors: Andrea SCHMIDT, Ludwigshafen (DE); Michael Bock, Ruppertsberg (DE); Roderich Roettger, Langenhagen (DE); Robert Baumann, Mannheim (DE); Axel Franzke, Mannheim (DE)

(21) Appl. No.: 13/693,281

(22) Filed: Dec. 4, 2012

Related U.S. Application Data
(60) Provisional application No. 61/566,711, filed on Dec. 5, 2011.

(51) Int. Cl. C07C 68/08; C07C 29/82

(52) U.S. Cl. CPC C07C 68/08; C07C 29/82
USPC 558/277

(57) ABSTRACT

The invention relates to a process for obtaining a dialkyl carbonate and an alkylene glycol from a stream comprising dialkyl carbonate, alkylene carbonate, alkylene glycol and alcohol, comprising the following steps:
(a) distillatively removing a stream (5) comprising dialkyl carbonate and alkylene glycol as a heteroazeotrope from the stream comprising dialkyl carbonate, alkylene carbonate, alkylene glycol and alcohol in a first distillation stage (1),
(b) separating the stream (5) comprising dialkyl carbonate and alkylene glycol as a heteroazeotrope into a first crude product stream (27) comprising essentially di-alkyl carbonate and a second crude product stream (29) comprising essentially alkylene glycol in an apparatus for phase separation (25).

The invention relates to a process for obtaining a dialkyl carbonate and an alkylene glycol from a stream comprising dialkyl carbonate, alkylene carbonate, alkylene glycol and alcohol.

The stream comprising dialkyl carbonate, alkylene carbonate, alkylene glycol and alcohol typically arises in the preparation of dialkyl carbonate as the main product and alkylene glycol as a by-product, by transesterification of a cyclic alkylene carbonate with alcohols in the presence of a catalyst.

The preparation of dialkyl carbonate from cyclic alkylene carbonate and alcohol, in which alkylene glycol simultaneously forms as a by-product, is known and has been described many times before. The synthesis is a reversible equilibrium reaction in which the product mixture of dialkyl carbonate and alkylene glycol always also comprises proportions of unconverted alkylene carbonate and alcohol. The transesterification reaction in a reactor with downstream purification of the products in several distillation columns is described, for example, in WO-A 2011/058168. In the transesterification reaction described here, it is only possible to attain the conversion possible at equilibrium. Unconverted starting materials have to be removed in maximum purity from the products since product recycled into the transesterification reaction unfavorably influences the equilibrium and reduces the productivity.

As well as the transesterification reaction in a separate reactor, a known alternative is to use a reactive distillation in which alcohol and alkylene carbonate are supplied, and transesterification and fractionation of the components proceeds simultaneously in a column. Such a reactive distillation is described, for example, in JP-A 2005/357704, U.S. Pat. No. 6,346,638, EP-A 1 967 507 or DE-A 10 2009 030 680. The distillative removal of the products in the reactive distillation favorably influences the equilibrium of the reaction. However, such a reactive distillation is possible only when the residence times needed for the reaction or the required conversion can be established in a column. A favorable influence on the residence time in the reactive distillation column can be achieved, for example, by suitable length/diameter ratios as described in US-A 2009/030223 or by particular distance ratios between the feed point of the alcohol and the mixture comprising alcohol and dialkyl carbonate as the stream recycled from the workup, as described in DE-A 10 2009 030 680. However, disadvantages are that the low boiler mixture which comprises dialkyl carbonate and is drawn off via the top also comprises unconverted alcohol, and that the alkylene glycol withdrawn from the bottom additionally also comprises unconverted alkylene carbonate. In these cases, the dialkyl carbonate is purified in at least one further distillation column. This division is energy-intensive and complex in apparatus terms, since the alcohol and the dialkyl carbonate which form generally form an azeotrope. The performance of such a distillation for separation is described, for example, in US-A 2005/0080287. Another alternative disclosed is to separate the mixture by extractive distillation, in which case the alkylene carbonate is used as the extractant. This is disclosed, for example, in US-A 2007/197816. The dialkyl carbonate is then removed from the extractant in a further distillation.

In order to remove the dialkyl carbonate from the equilibrium as early as during the reaction, U.S. Pat. No. 5,489,703 discloses adding the extractant as early as within the reaction zone. If unconverted alcohol is to be recycled into the synthesis, a portion of the product is included in this case too. However, the recycling of the product into the transesterification reaction has an unfavorable effect on the equilibrium since the formation of the starting material is promoted and hence the productivity is worsened. In order to obtain a higher conversion, U.S. Pat. No. 5,847,189 proposes a construction in which the reactor is on the outside, and therefore the supply of a mixture comprising alcohol and dialkyl carbonate can also lead to high conversions. However, a downstream distillative purification of the alcohol-dialkyl carbonate azeotrope is needed here too in order to obtain the product.

In all processes in which an alkylene glycol is obtained as a by-product which are known from the prior art, a further purification step is needed, in which the alkylene glycol is removed from the crude product and unconverted alkylene carbonate is recycled into the synthesis. The workup of a mixture comprising alkylene carbonate and alkylene glycol at high temperatures with long residence times, especially in the presence of a catalyst as used for the transesterification reaction, however, causes side reactions to give polyalkylene glycols such as dialkylene glycol and trialkylene glycol. In order to avoid these side reactions, it is necessary to perform the distillation under moderate conditions under reduced pressure which unavoidably result in formation of an alkylene carbonate-alkylene glycol azeotrope. This azeotrope comprises a high alkylene glycol content. Simultaneous recycling of unconverted alkylene carbonate together with alkylene glycol, however, again has an unfavorable effect on the equilibrium of the transesterification reaction and reduces the productivity.

The workup of a mixture comprising alkylene carbonate and alkylene glycol and the preparation of a high-purity alkylene glycol is described, for example in U.S. Pat. No. 5,847,189. For workup, the high boiler mixture comprising alkylene carbonate and a water stream are passed together continuously into a hydrolysis reactor, and the alkylene glycol formed is withdrawn continuously. The supply of water results in decomposition of the alkylene carbonate to alkylene glycol and carbon dioxide.

As an alternative, US-A 2003/0078448 describes a process for purifying alkylene glycol, in which the mixture comprising alkylene carbonate and alkylene glycol is first distilled and separated into an alkylene carbonate-alkylene glycol azeotrope fraction and pure alkylene glycol. The alkylene carbonate-alkylene glycol azeotrope is converted by hydrolysis of alkylene carbonate to pure alkylene glycol and carbon dioxide. A disadvantage of the hydrolysis is, however, that the alkylene carbonate decomposes and hence can no longer be converted to the dialkyl carbonate product of value. This portion is obtained as carbon dioxide and is lost from the process.

In order to avoid the hydrolysis, JP-A 2006/023065 discloses performing a multistage purification in which the bottom stream comprising alkylene glycol and alkylene carbonate is distilled in a further column and the alkylene glycol is obtained from a side draw.

U.S. Pat. No. 6,479,689 describes, for purification of the alkylene glycol, an ether-forming reaction between cyclic alkylene carbonate and portions of the alkylene glycol. The reaction is followed by the separation in a column into a carbon dioxide-comprising low-boiler mixture which is removed via the top, and into a bottom fraction comprising alkylene glycol. The carbon dioxide is stripped out of the low boiler mixture with nitrogen in a further column, and the low boiler mixture comprising alcohol with or without dialkyl carbonate is returned to the reactive distillation column. This embodiment, however, likewise requires a multistage workup sequence for purification of the alkylene glycol and results in a loss of alkylene carbonate as the ether compound.

In all processes known from the prior art, it is disadvantageous that the separation of the product mixture originating from the transesterification can be performed only in an energy-intensive manner with complex apparatus. This is the case especially when the dialkyl carbonate forms an azeotrope with the alcohol used. The result of this is that, when the starting materials are recycled into the synthesis without additional complex separating operations, a portion of the product is always also returned, thus unfavorably influencing the transesterification reaction. In addition, in the processes known from the prior art, the separation of the homoazeotropic mixture of unconverted alkylene carbonate and the alkylene glycol formed is possible only by decomposition or reaction and hence loss of alkylene carbonate, or multistage and thus energy-intensive distillation.

It is therefore an object of the present invention to provide a continuous process for preparing dialkyl carbonates, which does not have the disadvantages known from the prior art and in which the products and unconverted reactants can be separated in an energetically favorable manner and with a lower level of apparatus complexity.

The object is achieved by a process for obtaining a dialkyl carbonate and an alkylene glycol from a stream comprising dialkyl carbonate, alkylene carbonate, alkylene glycol and alcohol, comprising the following steps:
(a) distillatively removing a stream comprising dialkyl carbonate and alkylene glycol as a heteroazeotrope from the stream comprising dialkyl carbonate, alkylene carbonate, alkylene glycol and alkylene alcohol in a first distillation stage,
(b) separating the stream comprising dialkyl carbonate and alkylene glycol as a heteroazeotrope into a first crude product stream comprising essentially dialkyl carbonate and a second crude product stream comprising essentially alkylene glycol in an apparatus for phase separation.

In the context of the present invention, “first crude product stream comprising essentially dialkyl carbonate” means that the proportion of dialkyl carbonate in this stream is at least 70% by weight, preferably at least 90% by weight and especially at least 95% by weight. As well as dialkyl carbonate, the first crude product stream generally also comprises residues of alkylene glycol.

“Second crude product stream comprising essentially alkylene glycol” means that this crude product stream comprises at least 70% by weight of alkylene glycol, more preferably at least 90% by weight of alkylene glycol and especially at least 95% by weight of alkylene glycol. As well as alkylene glycol, the second crude product stream generally also comprises residues of dialkyl carbonate.

The process according to the invention and the separation of the stream comprising dialkyl carbonate and alkylene glycol as a heteroazeotrope by means of an apparatus for phase separation make it possible to separate the product mixture with a lower level of apparatus complexity and lower energy requirement than in the processes known from the prior art.

In order to be able to perform the process according to the invention, it is necessary that the dialkyl carbonate is prepared using an alcohol which forms, as a result of the transesterification of the alkylene carbonate, a dialkyl carbonate which forms a heteroazeotrope together with the alkylene glycol which forms. The formation of the heteroazeotrope enables separation in an apparatus for phase separation without energy expenditure. It is additionally preferable when the alcohol used does not form an azeotrope with the dialkyl carbonate which forms in the reaction.

The stream which comprises dialkyl carbonate, alkylene carbonate, alkylene glycol and alcohol, and from which the stream comprising dialkyl carbonate and alkylene glycol as a heteroazeotrope is removed by distillation, originates from a transesterification reaction in which the alkylene carbonate is transesterified with the alcohol to give dialkyl carbonate. The alkylene glycol is obtained as a by-product.

The transesterification is generally performed under homogeneous or heterogeneous catalysis. In the case of homogeneous catalysis, the catalyst is likewise present in the stream comprising dialkyl carbonate, alkylene carbonate, alkylene glycol and alcohol. Suitable catalysts are known to those skilled in the art and are described, for example in DE-A 10 2009 053 370, EP-A 1 961 721 and WO-A 2011/058168. Examples of suitable homogeneous catalysts include an alkali metal, an alkali metal salt of an organic acid such as acetic acid, propionic acid, butyric acid, benzoic acid, stearic acid, a hydride, oxide, hydroxide, alkoxide, amide, carbonate or dicarbonate of an alkali metal or an alkali metal salt, which derives from an inorganic acid, for example hydrochloric acid, hydrobromic acid or hydriodic acid, nitric acid, sulfuric acid, hydrofluoric acid, phosphoric acid, hydrogen cyanide, hydrocyanic acid or thiocyanic acid.

Preference is given to using potassium hydroxide, sodium hydroxide or the potassium or sodium salt of the alcohol used in the transesterification.

The amount of homogeneous catalyst is typically less than 10% by weight, generally in the range from 0.0001 to 5% by weight and especially in the range from 0.001 to 2% by weight, in each case based on the amount of the reaction mixture.

In principle, it is also possible to perform the transesterification under heterogeneous catalysis. The catalysis can be executed either in an upstream reactor or in the first distillation stage. Suitable heterogeneous catalysts are, for example, ionic exchange resins with functional groups formed from tertiary amines, quaternary ammonium groups, in which case examples of counterions include chloride, hydrogensulfate or hydroxide, ammonium-exchanged zeolites or alkali metal or alkaline earth metal silicates impregnated on silicon dioxide supports.

The reaction can be performed in a separate reactor, in which case alkylene carbonate and alcohol and catalyst are supplied to the reactor and the transesterification is performed in the reactor. The stream comprising dialkyl carbonate, alkylene carbonate, alkylene glycol and alcohol is then withdrawn from the reactor and supplied to the first distillation stage in step (a). Alternatively, it is also possible that the first distillation stage in step (a) comprises a reactive distillation to which alkylene carbonate and alcohol are added as reactants and are converted in an equilibrium reaction to dialkyl carbonate and alkylene glycol, giving the stream comprising dialkyl carbonate, alkylene carbonate, alkylene glycol and monoalcohol. In the reactive distillation column, the stream comprising alkylene glycol and dialkyl carbonate as a heteroazeotrope is then removed from this stream.

In a first embodiment, the first distillation stage comprises a dividing wall column in which the stream comprising dialkyl carbonate, alkylene carbonate, alkylene glycol and alcohol is separated into an alcohol-comprising top stream, a stream which comprises alkylene carbonate and alkylene glycol and forms a homoazeotrope as the bottom stream, and the stream which comprises dialkyl carbonate and alkylene glycol as a heteroazeotrope and is drawn off as a side stream. The alcohol-comprising top stream can be recycled directly into the reactor or into the reactive distillation column as a reactant. The bottom stream comprises, as well as alkylene carbonate and alkylene glycol which form the homoazeotrope, generally additionally also the catalyst used for the transesterification. The stream which comprises dialkyl carbonate and alkylene glycol as a heteroazeotrope and is withdrawn from the dividing wall column via a side draw is catalyst-free.

Alternatively to the dividing wall column, it is also possible that the first distillation stage comprises two distillation columns, in which case the stream comprising dialkyl carbonate, alkylene carbonate, alkylene glycol and alcohol is separated into a top stream comprising dialkyl carbonate, alkylene glycol and alcohol, and a homoazeotrope-forming bottom stream comprising alkylene carbonate and alkylene glycol. In a second distillation column, the top stream comprising dialkyl carbonate, alkylene glycol and alcohol from the first distillation column is separated into a top stream comprising the alcohol and the stream comprising dialkyl carbonate and alkylene glycol as a heteroazeotrope as the bottom stream.

The stream which is obtained as a side draw from the dividing wall column or as the bottom stream from the second distillation column and comprises dialkyl carbonate and alkylene glycol as a heteroazeotrope is then sent to the separation in an apparatus for phase separation in step (b).

In a preferred embodiment, the stream which comprises homoazeotrope-forming alkylene carbonate and alkylene glycol, is obtained in the first distillation stage and is obtained either as the bottom stream of the dividing wall column or as the bottom stream of the first distillation column is sent to a second distillation stage. Additionally supplied to the second distillation stage is a portion of the first crude product stream comprising dialkyl carbonate from step (b) as an entraining agent. A bottom stream comprising alkylene carbonate and top stream comprising dialkyl carbonate and alkylene glycol are then withdrawn from the second distillation stage. The bottom stream comprising alkylene carbonate can then likewise be recycled into the transesterification as a reactant. If the transesterification is performed under homogeneous catalysis, the homoazeotrope-forming stream which comprises alkylene carbonate and alkylene glycol and is sent to the second distillation stage additionally also comprises the catalyst. This is likewise obtained at the bottom of the second distillation stage and can be recycled into the transesterification together with the alkylene carbonate. The top stream comprising dialkyl carbonate and alkylene glycol is supplied to the apparatus for phase separation in step (b), such that dialkyl carbonate and the alkylene glycol are obtained as products. As a result of this procedure, the unconverted alkylene carbonate is recycled into the transesterification without losses.

If the first distillation stage is performed in a dividing wall column, the dividing wall column is preferably operated at a pressure in the range from 0.01 to 0.5 bar, preferably in the range from 0.02 to 0.2 bar and more preferably in the range from 0.03 to 0.1 bar. The temperature at the bottom of the dividing wall column is preferably in the range from 120 to 200° C., more preferably in the range from 130 to 190° C. and especially in the range from 140 to 180° C. and the temperature at the top of the dividing wall column in the range from 10 to 100° C., more preferably in the range from 20 to 80° C. and especially in the range from 25 to 60° C.

The pressure figures given above and below are absolute pressure figures, unless explicitly stated otherwise.

If the first distillation stage is performed in two distillation columns, the first distillation column is preferably operated at a pressure in the range from 0.01 to 0.5 bar, more preferably in the range from 0.01 to 0.1 bar and especially in the range from 0.01 to 0.05 bar, the bottom temperature being preferably in the range from 120 to 200° C., more preferably in the range from 130 to 190° C. and especially in the range from 150 to 185° C. and the top temperature in the range from 20 to 100° C., more preferably in the range from 30 to 80° C. and especially in the range from 40 to 70° C. The pressure in the second distillation column is preferably at a pressure in the range from 0.05 to 0.5 bar, more preferably in the range from 0.1 to 0.3 bar and especially in the range from 0.1 to 0.2 bar. The temperature at the bottom of the second distillation column is preferably in the range from 80 to 200° C., more preferably in the range from 100 to 150° C. and especially in the range from 110 to 130° C., and the temperature at the top of the second distillation column in the range from 10 to 100° C., more preferably in the range from 20 to 80° C. and especially in the range from 25 to 70° C.

The second distillation stage, in which the homoazeotrope of alkylene carbonate and alkylene glycol is separated in the presence of the dialkyl carbonate as an entraining agent, is preferably operated at a pressure in the range from 0.01 to 0.5 bar, more preferably in the range from 0.02 to 0.2 bar and especially in the range from 0.05 to 0.1 bar, the bottom temperature being preferably in the range from 100 to 200° C., more preferably in the range from 130 to 190° C. and especially in the range from 140 to 180° C., and the top temperature in the range from 50 to 150° C., more preferably in the range from 80 to 120° C. and especially preferably in the range from 90 to 110° C.

In an alternative embodiment, the first distillation stage comprises a dividing wall column in which the stream comprising dialkyl carbonate, alkylene carbonate, alkylene glycol and alcohol is separated into a bottom stream comprising alkylene carbonate, a top stream comprising alcohol, and the stream which comprises dialkyl carbonate and alkylene glycol as a heteroazeotrope and is drawn off as a side stream. Alternatively, the first distillation stage in this case comprises a first distillation column in which the stream comprising dialkyl carbonate, alkylene carbonate, alkylene glycol and alcohol is separated into a bottom stream comprising alkylene carbonate and a top stream comprising dialkyl carbonate, alcohol and alkylene glycol, and a second distillation column in which the top stream comprising dialkyl carbonate, alcohol and alkylene glycol from the first distillation column is separated into an alcohol-comprising top stream and the stream comprising dialkyl carbonate and alkylene glycol as a heteroazeotrope as the bottom stream. Irrespective of whether the first distillation stage is conducted in a dividing wall column or in two distillation columns, a portion of the first crude product stream comprising dialkyl carbonate from step (b) is additionally added thereto as an entraining agent. The addition of the first crude product stream comprising dialkyl carbonate as an entraining agent breaks the homoazeotrope of alkylene carbonate and alkylene glycol, such that the alkylene carbonate can be removed in alkylene glycol-free form and recycled into the reaction. This avoids any adverse effect of alkylene glycol recycled into the reaction on the equilibrium. In addition, this achieves freedom from alkylene carbonate in the top stream comprising alkylene glycol, and no passage of alkylene carbonate into the downstream process stages.

If a portion of the first crude product stream comprising dialkyl carbonate from step (b) is added to the first distillation stage as an entraining agent, it is possible to add the portion of the first crude product stream comprising dialkyl carbonate to the feed to the first distillation stage. Alternatively, it is also possible to supply the portion of the first crude stream comprising dialkyl carbonate as a separate feed to the dividing wall column or the first distillation column, in which case the portion of the first crude product stream comprising dialkyl carbonate is added on the feed side of the dividing wall column or to the first distillation column, above the feed of alkylene carbonate and alcohol, or of the stream comprising dialkyl carbonate, alkylene glycol, alkylene carbonate and alcohol.

In order to improve the quality of the product, it is additionally preferred when the first crude product stream which comprises essentially dialkyl carbonate and is withdrawn from the apparatus for phase separation in step (b), is supplied to a distillative operation in which the alkylene glycol still present in the first crude product stream comprising essentially dialkyl carbonate is removed therefrom. The alkylene glycol removed from the first crude product stream comprising essentially dialkyl carbonate generally still comprises dialkyl carbonate and is therefore preferably recycled into the apparatus for phase separation in step (b). This makes it possible likewise to obtain the dialkyl carbonate removed in the distillation as a product.

The distillation stage for removal of the alkylene glycol from the first crude product stream comprising essentially dialkyl carbonate is preferably performed at a pressure in the range from 0.05 to 1 bar, more preferably at a pressure in the range from 0.09 to 0.5 bar and especially at a pressure in the range from 0.1 to 0.2 bar. The top temperature is preferably in the range from 10 to 150° C., more preferably in the range from 50 to 145° C. and especially in the range from 90 to 140° C. and the bottom temperature in the range from 80 to 200° C., more preferably in the range from 90 to 160° C. and especially in the range from 95 to 140° C.

In order likewise to be able to obtain the dialkyl carbonate present in the second crude product stream comprising alkylene glycol as a product, the second crude product stream comprising essentially alkylene glycol is preferably also sent to a distillative operation in which the dialkyl carbonate still present in the crude product stream comprising essentially alkylene glycol is removed therefrom. Since the dialkyl carbonate is generally removed in the form of an azeotrope with alkylene glycol present therein, it is also preferable here to recycle the dialkyl carbonate removed into the apparatus for phase separation in step (b). This makes it possible to obtain the alkylene glycol removed in the distillation likewise as a product.

The distillation for processing of the second crude product stream comprising essentially alkylene glycol is conducted preferably at a pressure in the range from 0.05 to 1 bar, more preferably at a pressure in the range from 0.09 to 0.3 bar and especially at a pressure in the range from 0.1 to 0.2 bar. The bottom temperature is preferably in the range from 80 to 200° C., more preferably in the range from 90 to 180° C. and especially in the range from 110 to 150° C. The top temperature is preferably in the range from 20 to 180° C., more preferably in the range from 50 to 170° C. and especially in the range from 80 to 160° C.

The apparatus for phase separation in step (b) may be any desired suitable liquid phase separator known to those skilled in the art, for example as mentioned in Perry's Chemical Engineers' Handbook, seventh edition, 1998, p. 15 to 26 to 15 to 27, Gravity Settlers, Decanters, and Liquid Extraction, second edition, McGraw-Hill Book Company 1963, p. 440 to 450.

The apparatus for phase separation is operated preferably at a pressure in the range from 1 to 5 bar, more preferably in the range from 1 to 3 bar and especially in the range from 1 to 2 bar and at a temperature in the range from 1 to 90° C., more preferably in the range from 1 to 50° C. and especially in the range from 5 to 40° C.

The process according to the invention can be performed continuously or batchwise. It is preferable to operate the process continuously. For this purpose, it is necessary, more particularly, to use a continuously operable apparatus for phase separation.

Alcohols suitable for the process according to the invention are those of the general formula (I)

R—OH (I)

in which R is:
R is linear or branched C₂-C₈-alkyl, which optionally also comprises rings, linear or branched C₃-C₈-alkenyl, linear or branched C₃-C₈-alkynyl or C₃-C₈-cycloalkyl,
where one or two non-terminal CH₂ groups are optionally replaced by heteroatoms from the group of O, and
where R except from C₂-alkyl is unsubstituted or substituted by 1 to 3, in the case of halogen up to the maximum possible number, of substituents from the group of halogen, C₁-C₄-alkyl, C₁-C₄-alkoxy, C₆-C₁₀-aryl, di-(C₁-C₄-alkyl)-amino and furylmethyl and for C₂-alkyl always is substituted by 1 to 3, in the case of halogen up to the maximum possible number, of substituents from the group of halogen, C₁-C₄-alkyl, C₁-C₄-alkoxy, C₆-C₁₀-aryl, di-(C₁-C₄-alkyl)-amino and furylmethyl.

In one embodiment, R is linear or branched C₂-C₈-alkyl.

In a preferred embodiment, R is linear or branched C₃-C₈-alkyl, which is unsubstituted or substituted by 1 methoxy substituent, particularly C₃-C₆-alkyl, which is unsubstituted or substituted by 1 methoxy substituent.

When R is branched C₂-C₈-alkyl, the branch is preferably in the β and/or γ position, more particularly in the β position.

When R is C₃-C₈-alkynyl, it is preferably propargyl.

When R is C₃-C₈-cycloalkyl, it is preferably cyclopentyl, cyclohexyl or cycloheptyl, especially cyclopentyl or cyclohexyl.

When non-terminal CH₂ groups are replaced by heteroatoms, one non-terminal CH₂ group is preferably replaced by an oxygen atom.

In a particular embodiment R is linear C₃-C₆-alkyl, which comprises an oxygen atom in the β position.

When R is substituted, preferably the β and/or γ position, more particularly the 13 position, is substituted

When R is substituted by a substituent from the group of halogen, it is preferably substituted by 1-8 fluorine or 1-3 chlorine substituents.

When R is substituted by substituents from the group of C₁-C₄-alkyl, it is preferably substituted by methyl or ethyl substituents, especially methyl substituents.

When R is substituted by substituents from the group of C₁-C₄-alkoxy, it is preferably substituted by 1 methoxy or ethoxy substituents.

Suitable alcohols are, for example 2,2,2-trifluoroethanol, 1,1,2,2-tetrafluoroethanol, 1,1,2,2,2-pentafluoroethanol, 1,1,1-trifluoro-2-propanol, 1,1-dimethyl-1-ethanol, 2-propanol, 2,2,3,3,4,4,4-heptafluoro-1-butanol, 2,2,3,3,3-pentafluoro-1-propanol, 1-propanol, 2-butanol, 1,1-dimethyl-1-propanol, isobutanol, 3-methyl-2-butanol, 2-propyn-1-ol, 2,2-dimethyl-1-propanol, 3-pentanol, 1-butanol, 2,3-dimethyl-2-butanol, 2-pentanol, 3,3-dimethyl-2-butanol, 2-methyl-2-pentanol, 3-methyl-3-pentanol, 2-chloro-1-propanol, 2-methyl-3-pentanol, 2-chloro-1-ethanol, 1-chloro-2-propanol, 2-methyl-1-butanol, 2,3,3-trimethyl-2-butanol, 3-methyl-1-butanol, 4-methyl-2-pentanol, 2,4-dimethyl-2-pentanol, 3-methyl-2-pentanol, 1-methylcyclopentanol, 3-hexanol, 2,2-dimethyl-3-pentanol, 2,2-dimethyl-1-butanol, 1-pentanol, 4,4-dimethyl-2-pentanol, 2,4-dimethyl-3-pentanol, 2,3-dimethyl-3-pentanol, 2,3-dimethyl-2-pentanol, 2-hexanol, cyclopentanol, 2-methyl-2-hexanol, 3-ethyl-3-pentanol, 3,3-dimethyl-1-butanol, 3-methyl-3-hexanol, 2-ethyl-1-butanol, 2-methyl-3-hexanol, 5-methyl-3-hexanol, 2-methyl-1-pentanol, 2,3-dimethyl-1-butanol, 2,4-dimethyl-2-hexanol, 5-methyl-2-hexanol, 3,4-dimethyl-3-hexanol, 4-methyl-1-pentanol, 3-methyl-2-hexanol, 3-ethyl-2-pentanol, 2,4-dimethyl-4-hexanol, 3-methyl-1-pentanol, 1-ethylcyclopentanol, 5-methyl-3-heptanol, 4-heptanol, 4-methyl-3-heptanol, 2,2-dimethyl-3-hexanol, 2-methyl-2-heptanol, 3-heptanol, 1-hexanol, 2,3-dimethyl-3-hexanol, 2-heptanol, 3-ethyl-3-hexanol, 2,5-dimethyl-3-hexanol, 4,4-dimethyl-1-pentanol, 2,4-dimethyl-3-hexanol, 3-methyl-3-heptanol, cyclohexanol, 3-chloro-1-propanol, 4-methyl-4-heptanol, cycloheptanol, 2,4-dimethyl-1-pentanol, 2-methyl-1-hexanol, 3-methyl-4-heptanol, cis-2-methylcyclohexanol, 3-methyl-2-heptanol, 2-methyl-4-heptanol, trans-2-methylcyclohexanol, 2-methyl-3-heptanol, 2,2,4-trimethyl-1-pentanol, 1-methylcyclohexanol, 1-ethylcyclohexanol, furfuryl alcohol, cis-4-methylcyclohexanol, trans-4-methylcyclohexanol, 4-methyl-2-heptanol, 5-methyl-2-heptanol, 5-methyl-1-hexanol, 3-methyl-1-hexanol, 6-methyl-2-heptanol, 2,4,4-trimethyl-1-pentanol, 4-methyl-1-hexanol, 3-octanol, cis-3-methylcyclohexanol, 1,3-dichloro-2-propanol, trans-3-methylcyclohexanol, 2,6-dimethylcyclohexanol, 2-methoxy-1-ethanol or 2-ethoxy-1-ethanol.

Preferred alcohols are 1-propanol, 1-butanol, 1-pentanol, 1-hexanol, isobutanol, 2-methyl-1-butanol, 3-methyl-1-butanol, or 2-methoxyethanol, especially preferred alcohols being 1-propanol, 1-butanol, 1-pentanol, isobutanol or 3-methyl-1-butanol.

Alkylene carbonates suitable for the process according to the invention are alkylene carbonates of the general formula (II)

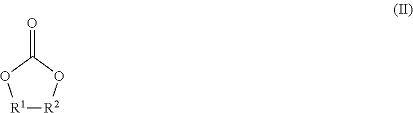

where R¹ and R² are each independently —(CR³R⁴)ₘ— where m=1-3, where R³ and R⁴ are each independently hydrogen, C₁-C₁₀-alkyl or C₆-C₁₀-aryl or both R⁴ radicals together form a C₅-C₁₀ ring.

The alkylene carbonates used are more preferably ethylene carbonate and propylene carbonate.

In the transesterification reaction, the ring structure of the alkylene carbonate is broken open and the R¹ and R² radicals of the alkylene carbonate are each substituted by the R radical of the alcohol used. In this case, it is also possible to use a mixture of several alcohols, such that the R¹ and R² radicals are substituted by different radicals of the alcohols.

The dialkyl carbonates formed in the transesterification are generally those of the general formula (III)

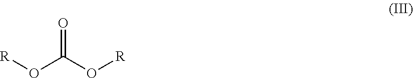

where R is the same or different and corresponds to the above definition of the R radical of the alcohol of the formula (I).

R is preferably the same.

The carbonates may be symmetric or else unsymmetric. The carbonates mentioned are a selection of the possible combinations of different alcohols.

Dialkyl carbonates preparable in the transesterification reaction are, for example, di(2,2,2-trifluoroethyl)carbonate, di(1,1,2,2-tetrafluoroethyl)carbonate, di(1,1,2,2,2-pentafluoroethyl)carbonate, di(1,1,1-trifluoro-2-propyl)carbonate, di-1,1-dimethyl-1-ethyl carbonate, di-2-propyl carbonate, di(2,2,3,3,4,4,4-heptafluoro-1-butyl)carbonate, di(2,2,3,3,3-pentafluoro-1-propyl)carbonate, di-1-propyl carbonate, di-2-butyl carbonate, di-1,1-dimethyl-1-propyl carbonate, diisobutyl carbonate, di(3-methyl-2-butyl)carbonate, di(2-propyn-1-ol) carbonate, di(2,2-dimethyl-1-propyl)carbonate, di-3-pentyl carbonate, di-1-butyl carbonate, di(2,3-dimethyl-2-butyl)carbonate, di-2-pentyl carbonate, di(3,3-dimethyl-2-butyl)carbonate, di-(2-methyl-2-pentyl)carbonate, di-(3-methyl-3-pentyl)carbonate, di(2-chloro-1-propyl)carbonate, di(2-methyl-3-pentyl)carbonate, di(2-chloro-1-ethyl)carbonate, di(1-chloro-2-propyl)carbonate, di(2-methyl-1-butyl)carbonate, di(2,3,3-trimethyl-2-butyl)carbonate, di(3-methyl-1-butyl)carbonate, di(4-methyl-2-pentyl)carbonate, di(2,4-dimethyl-2-pentyl)carbonate, di-(3-methyl-2-pentyl)carbonate, di(1-methylcyclopentyl)carbonate, di(3-hexyl)carbonate, di(2,2-dimethyl-3-pentyl)carbonate, di(2,2-dimethyl-1-butyl)carbonate, di-1-pentyl carbonate, di(4,4-dimethyl-2-pentyl)carbonate, di(2,4-dimethyl-3-pentyl)carbonate, di(2,3-dimethyl-3-pentyl)carbonate, di(2,3-dimethyl-2-pentyl)carbonate, di-2-hexyl carbonate, dicyclopentyl carbonate, di(2-methyl-2-hexyl)carbonate, di(3-ethyl-3-pentyl)carbonate, di(3,3-dimethyl-1-butyl)carbonate, di(3-methyl-3-hexyl)carbonate, di(2-ethyl-1-butyl)carbonate, di(2-methyl-3-hexyl)carbonate, di(5-methyl-3-hexyl)carbonate, di(2-methyl-1-pentyl)carbonate, di(2,3-dimethyl-1-butyl)carbonate, di(2,4-dimethyl-2-hexyl)carbonate, di(5-methyl-2-hexyl)carbonate, di(3,4-dimethyl-3-hexyl)carbonate, di(4-methyl-1-pentyl)carbonate, di(3-methyl-2-hexyl)carbonate, di(3-ethyl-2-pentyl)carbonate, di(2,4-dimethyl-4-hexyl)carbonate, di(3-methyl-1-pentyl)carbonate, di(1-ethylcyclopentyl)carbonate, di(5-methyl-3-heptyl)carbonate, di(4-heptyl)carbonate, di(4-methyl-3-heptyl)carbonate, di(2,2-dimethyl-3-hexyl)carbonate, di(2-methyl-2-heptyl)carbonate, di(3-heptyl)carbonate, di(1-hexyl)carbonate, di(2,3-dimethyl-3-hexyl)carbonate, di(2-heptyl)carbonate, di(3-ethyl-3-hexyl)carbonate, di(2,5-dimethyl-3-hexyl)carbonate, di(4,4-dimethyl-1-pentyl)carbonate, di(2,4-dimethyl-3-hexyl)carbonate, di(3-methyl-3-heptyl)carbonate, di(cyclohexyl)carbonate, di(3-chloro-1-propyl)carbonate, di(4-methyl-4-heptyl)carbonate, di(cycloheptyl)carbonate, di(2,4-dimethyl-1-pentyl)carbonate, di(2-methyl-1-hexyl)carbonate, di(3-methyl-4-heptyl)carbonate, di(cis-2-methylcyclohexyl)carbonate, di(2-methylcyclohexyl)carbonate, di(3-methyl-2-heptyl)carbonate, di(2-methyl-4-heptyl)carbonate, di(trans-2-methylcyclohexyl)carbonate, di(3-hydroxy-2-methyl propionaldehyde) carbonate, di(2-methyl-heptyl-3) carbonate, di(2,2,4-trimethyl-1-pentyl)carbonate, di(1-methylcyclohexyl)carbonate, di(1-ethylcyclohexanol) carbonate, di(2-hexyn-1-ol) carbonate, di(2-furanmethyl)carbonate, di(cis-4-methylcyclohexyl)carbonate, di(trans-4-methylcyclohexyl)carbonate, di(4-methyl-2-heptyl)carbonate, di(5-methyl-2-heptyl)carbonate, di(5-methyl-1-hexyl)carbonate, di(3-methyl-1-hexyl)carbonate, di(6-methyl-2-heptyl)carbonate, di(2,4,4-trimethyl-1-pentyl)carbonate, di(4-methyl-1-hexyl)carbonate, di(3-octyl)carbonate, di(cis-3-methylcyclohexyl)carbonate, di(1,3-dichloro-2-propyl)carbonate, di(trans-3-methylcyclohexyl)carbonate, di(2,6-dimethylcyclohexyl)carbonate, di-2-methoxyethyl carbonate or di-2-ethoxyethyl carbonate.

Preferred dialkyl carbonates are di-1-propyl carbonate, di-1-butyl carbonate, di-1-pentyl carbonate, di-1-hexyl carbonate, diisobutyl carbonate, di-2-methyl-1-butyl carbonate, di-3-methylbutyl carbonate or di-2-methoxyethyl carbonate.

The dialkyl carbonate is more preferably di-1-propyl carbonate, di-1-butyl carbonate, di-1-pentyl carbonate, diisobutyl carbonate, di-3-methyl-1-butyl carbonate or di-2-methoxyethyl carbonate.

The R¹ and R² radicals released in the transesterification with the hydroxyl groups of the alcohols used form an alkylene glycol of the general formula (IV)

HO—R¹R²—OH (IV).

The R¹ and R² radicals here are the same as described above for the alkylene carbonates.

When a stream comprising dialkyl carbonate, alkylene carbonate, alkylene glycol and alcohol is supplied to the process, it generally comprises 0.5 to 70% by weight of dialkyl carbonate, 1 to 50% by weight of alkylene carbonate, 1 to 40% by weight of alkylene glycol and 10 to 95% by weight of alcohol. The stream more preferably comprises 1 to 50% by weight of dialkyl carbonate, 2 to 30% by weight of alkylene carbonate, 2 to 30% by weight of alkylene glycol and 20 to 90% by weight of alcohol. More particularly, the stream comprises 5 to 40% by weight of dialkyl carbonate, 5 to 30% by weight of alkylene carbonate, 2 to 15% by weight of alkylene glycol and 40 to 80% by weight of alcohol.

In addition, the stream, especially in the case of a homogeneously catalyzed transesterification reaction, may comprise less than 10% by weight, more preferably 5 to 0.0001% by weight and especially 2 to 0.001% by weight of catalyst.

Working examples of the invention are shown in the figures and are illustrated in detail in the description which follows.

The figures show:

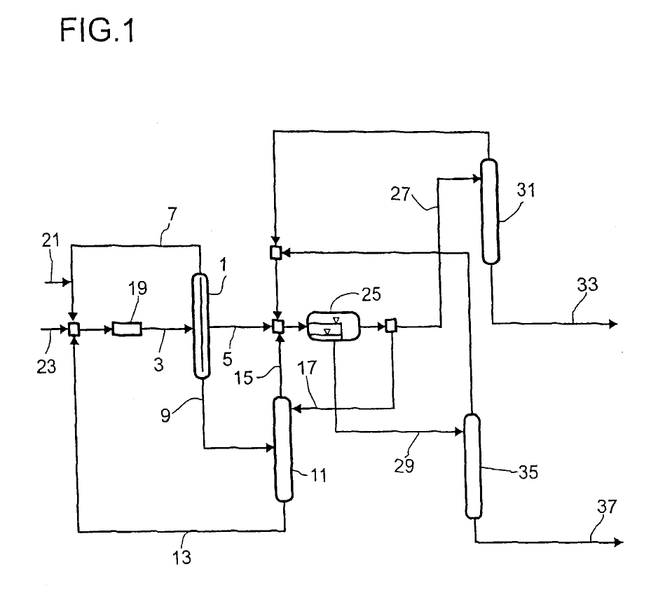

FIG. 1 a flow diagram of the process according to the invention in a first embodiment;

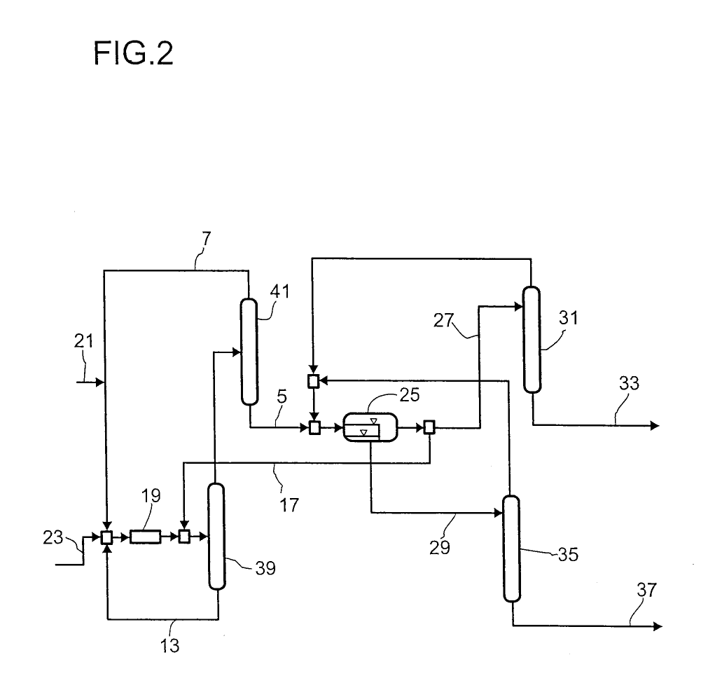

FIG. 2 a flow diagram of the process according to the invention in a second embodiment.

FIG. 1 shows a flow diagram of the process according to the invention in a first embodiment.

A stream which comprises dialkyl carbonate, alkylene carbonate, alkylene glycol and alcohol and is to be processed is sent to a first distillation stage. The first distillation stage comprises, in the embodiment shown in FIG. 1, a dividing wall column 1. The feed of the stream comprising dialkyl carbonate, alkylene carbonate, alkylene glycol and alcohol is configured as a side feed 3 to the dividing wall column 1. In the dividing wall column 1, a stream comprising dialkyl carbonate and alkylene glycol as a heteroazeotrope is removed by distillation. This stream is withdrawn in a side draw 5. At the top of the dividing wall column, an alcohol-comprising top stream 7 is obtained. At the bottom of the dividing wall column 1 a stream which comprises alkylene carbonate and alkylene glycol and forms a homoazeotrope is withdrawn as bottom stream 9. The homoazeotrope-forming stream comprising alkylene carbonate and alkylene glycol withdrawn as bottom stream 9 is sent to a second distillation stage 11. The second distillation stage 11 comprises a distillation column. In the second distillation stage 11, the stream comprising alkylene carbonate and alkylene glycol is separated into a bottom stream 13 comprising alkylene carbonate and a top stream 15 comprising alkylene glycol and dialkyl carbonate. In order to separate the mixture of alkylene carbonate and alkylene glycol present as a homoazeotrope in the second distillation stage 11, dialkyl carbonate is additionally supplied to the second distillation stage 11. In the embodiment described here, the supply is via a side feed 17 above the feed 9 of the mixture of alkylene glycol and alkylene carbonate. The alkylene carbonate obtained at the bottom of the second distillation stage 11 is, like the alcohol obtained at the top of the dividing wall column 1, recycled into a reactor 19 in which the alkylene carbonate and the alcohol react in a transesterification reaction to give dialkyl carbonate and alkylene glycol. The reaction is an equilibrium reaction, and so the stream comprising dialkyl carbonate, alkylene carbonate, alkylene glycol and alcohol is withdrawn from reactor 19 and recycled into the dividing wall column 1. Alcohol converted in the reaction is supplied to the reactor 19 via a first feed 21, and converted alkylene carbonate via a second feed 23. The alcohol can, as shown in FIG. 1, be supplied to the reflux of the top stream 7, and the alkylene carbonate to the reflux of the alkylene carbonate from the second distillation stage 11. It is also possible to supply each of the individual streams separately to the reactor. However, it is preferable to mix all streams supplied to the reactor 19 at a mixing point and to introduce them together into the reactor 19. The mixing can be effected in a simple manner by pipelines opening into a central supply line into reactor 19. The stream which comprises dialkyl carbonate and alkylene carbonate in the form of the heteroazeotrope and is withdrawn from the dividing wall column 1 as side draw 5 is supplied to an apparatus for phase separation 25. The apparatus used for phase separation 25 may, for example be a settler. In the apparatus for phase separation 25, the heteroazeotrope of dialkyl carbonate and alkylene glycol is separated into a first crude product stream 27 comprising essentially dialkyl carbonate and a second crude product stream 29 comprising essentially alkylene glycol. A portion of the first crude product stream 27 comprising essentially dialkyl carbonate is branched off and is passed via the feed for dialkyl carbonate 17 into the second distillation stage 11.

The first crude product stream 27 comprising essentially dialkyl carbonate is passed into a distillation column 31 for further processing. In the distillation column 31 the first crude product stream comprising essentially dialkyl carbonate is separated by distillation into a product stream 33 comprising dialkyl carbonate, which is withdrawn from the distillation column 31 as the bottom stream, and a top stream comprising essentially dialkyl carbonate, which is recycled into the apparatus for phase separation 25. The alkylene glycol removed in the distillation column 31 is present as an impurity in the first crude product stream 27 comprising essentially dialkyl carbonate.

Since the second crude product stream 29 comprising essentially alkylene glycol also comprises dialkyl carbonate as an impurity, it is likewise supplied to a distillation column 35. In the distillation column 35 a product stream 37 comprising alkylene glycol is removed, and a stream comprising essentially dialkyl carbonate is recycled into the apparatus for phase separation 25. It is possible here to mix the recycle streams from distillation columns 31, 35 and to recycle them together into the apparatus for phase separation 25. The recycled streams from distillation columns 31, 35 can additionally also be mixed with the stream which comprises dialkyl carbonate and alkylene glycol as the heteroazeotrope and is removed from dividing wall column 1. Likewise recycled into the apparatus for phase separation 25 is the top stream which comprises alkylene glycol and dialkyl carbonate and is drawn off at the top of the second distillation stage 11. The streams supplied to the apparatus for phase separation 25 in each case can be mixed before being fed in or be supplied as separate feeds. It is also possible to mix individual streams and to provide several feeds.

Alternatively to the embodiment shown in FIG. 1, in which the first distillation stage is configured in the form of a dividing wall column 1, it is also possible to replace the dividing wall column 1 with two distillation columns. In this case, the stream comprising dialkyl carbonate, alkylene glycol, alkylene carbonate and alcohol is supplied to the first distillation column and separated into a top stream comprising dialkyl carbonate, alkylene glycol and alcohol, and a homoazeotrope-forming bottom stream comprising alkylene carbonate and alkylene glycol, the homoazeotrope-forming bottom stream comprising alkylene carbonate and alkylene glycol being supplied to the second distillation stage. The top stream comprising dialkyl carbonate, alkylene glycol and alcohol is passed into a second distillation column of the first distillation stage, in which this stream is separated into the stream which comprises dialkyl carbonate and alkylene glycol as a heteroazeotrope and is supplied to the apparatus for phase separation 25 as the bottom stream, and the alcohol-comprising top stream which is recycled into the reactor 19.

It is additionally possible, instead of the separate reactor 19, to provide a reactive distillation, in which case the alcohol and the alkylene carbonate are supplied to the reactive distillation, and the stream which comprises dialkyl carbonate, alkylene carbonate, alkylene glycol and alcohol and is to be separated is produced and separated directly in the reactive distillation column.

FIG. 2 shows the process according to the invention in an alternative embodiment.

Differences in the embodiments shown in FIG. 2 from the embodiments shown in FIG. 1 include the fact that a portion of the crude product stream 27 comprising dialkyl carbonate is supplied to the first distillation stage in the form of stream 17 as an entraining agent. As a result of this, it is already possible in the first distillation column 39 of the first distillation stage to remove a stream 13 comprising alkylene carbonate, and no homoazeotrope of alkylene glycol and alkylene carbonate is obtained. It is therefore possible to dispense with the second distillation stage 11. FIG. 2 shows the first distillation stage with two distillation columns 39 and 41. As an alternative it is also possible to replace the two distillation columns 39 and 41 shown in FIG. 2 with a dividing wall column 1. In this case the stream comprising dialkyl carbonate is supplied to the dividing wall column in the feed, and the stream comprising dialkyl carbonate and alkylene glycol as a heteroazeotrope is not obtained as bottoms from a second distillation column, but rather, as shown in FIG. 1, as a side draw from the dividing wall column.

### EXAMPLES

Percentages given in the examples are—unless designated otherwise—percentages by weight.

### Example 1

2.63 kg/h of ethylene carbonate are reacted with 6.65 kg/h of isobutanol (molar ratio of isobutanol to ethylene carbonate 3:1) at 140° C. in the presence of 0.5 mol % (based on ethylene carbonate) of sodium isobutoxide as a catalyst in a tubular reactor to give 2.5 kg/h of diisobutyl carbonate and 0.93 kg/h of monoethylene glycol.

The reaction output is passed into a dividing wall column which is operated at a top pressure of 80 mbar (absolute). The distillation is conducted continuously at a bottom temperature of 170° C. and a reflux ratio of 0.76 g/g.

2.38 kg/h of liquid bottom product are obtained with a composition of 57.5% of ethylene carbonate, 20.5% monoethylene glycol, 15.1% diisobutyl carbonate and 0.55% sodium isobutoxide. The remainder consists predominantly of higher ethylene glycols.

At the top, 7.97 kg/h of a vapor stream are obtained with a composition of 99.91% isobutanol, 100 ppm of diisobutyl carbonate and water, which is condensed and recycled into the reactor, or into the column as reflux. A portion of the stream is discharged to remove water from the system.

In the side draw of the dividing wall column, 2.58 kg/h of a liquid biphasic stream are withdrawn, which comprises a composition of 83% diisobutyl carbonate and 17% monoethylene glycol. This stream is supplied to a phase separator. The phase separation into a diisobutyl carbonate-rich phase with 98.5% diisobutyl carbonate and a monoethylene glycol-rich phase with 97.6% monoethylene glycol is conducted at 40° C.

3.15 kg/h of the diisobutyl carbonate-rich phase are conducted into a column and distilled at a bottom temperature of 125° C. and a top pressure of 130 mbar. 2.5 kg/h of liquid bottom product are obtained with a composition of 99.94% diisobutyl carbonate and 600 ppm of monoethylene glycol. At the top, 0.65 kg/h of vapor stream with a composition of 2.2% isobutanol, 4.9% monoethylene glycol and 92.9% diisobutyl carbonate are withdrawn, condensed and recycled into the phase separation. 2.82 kg/h of the diisobutyl carbonate-rich phase are passed into the upper part of a column for separation of the bottom stream comprising monoethylene glycol and ethylene carbonate from the dividing wall column. The column is operated at a top pressure of 70 mbar and a bottom temperature of 159° C. 1.53 kg/h of a liquid bottom stream are obtained with the composition of 89.2% ethylene carbonate and 0.9% catalyst, and also higher ethylene glycols, which is recycled into the reactor. A portion of the bottom stream is discharged in order to remove higher ethylene glycols from the system.

At the top of the column, 3.67 kg/h of a vapor stream are obtained with the composition of 85.6% diisobutyl carbonate, 14.0% monoethylene glycol and 0.4% isobutanol, which is condensed and fed to the phase separation.

The monoethylene glycol-rich phase is purified by distillation at a mass flow rate of 0.95 kg/h in a column at a top pressure of 130 mbar and a bottom temperature of 139° C. This gives 0.93 kg/h of a liquid bottom stream with the composition of 99.9% monoethylene glycol and 0.1% ethylene carbonate.

At the top, 0.03 kg/h of a vapor stream is withdrawn with the composition of 39% isobutanol, 40.1% diisobutyl carbonate and 20.9% monoethylene glycol, condensed and recycled into the phase separation.

### Example 2

The reaction takes place under the same conditions as described in example 1. The reaction output is passed together with 4 kg/h of the diisobutyl carbonate-rich phase from the phase separator into a distillation column which is operated at a top pressure of 25 mbar. The distillation is operated continuously at a bottom temperature of 178° C. and a reflux ratio of 0.44 g/g.

At the bottom of the column, 2.17 kg/h of a liquid bottom stream are obtained with a composition of 92.3% ethylene carbonate, 0.3% diisobutyl carbonate, 0.49% sodium isobutoxide and residual higher ethylene glycols, which is recycled into the reactor.

At the top of the column, 15 kg/h of a vapor stream are obtained with a composition of 35.1% isobutanol, 57.5% diisobutyl carbonate and 7.4% monoethylene glycol, which is condensed and passed into a column for separation of isobutanol and the diisobutyl carbonate-monoethylene glycol azeotrope.

The distillation in the column is effected at a top pressure of 200 mbar and a bottom temperature of 124° C. This gives 4.1 kg/h of a vapor stream with a composition of 99% isobutanol, 0.9% diisobutyl carbonate and water, which is condensed and recycled into reactor (1). A portion of the top stream is discharged to remove water from the system.

At the bottom of the column, 6.79 kg/h of a biphasic bottom stream are obtained with a composition of 87.7% diisobutyl carbonate, 11.3% monoethylene glycol and 1% isobutanol, which is passed into a phase separator. The phase separation into a diisobutyl carbonate-rich phase with 97.02% diisobutyl carbonate and into a monoethylene glycol-rich phase with 94.5% monoethylene glycol is conducted at 40° C.

2.57 kg/h of the diisobutyl carbonate-rich phase are conducted into a column and distilled at a bottom temperature of 124° C. and a top pressure of 130 mbar. This gives 2.0 kg/h of liquid bottom product with a composition of 99.99% diisobutyl carbonate and 30 ppm of ethylene carbonate. At the top, 0.57 kg/h of vapor stream are withdrawn with the composition of 7.6% isobutanol, 87.2% diisobutyl carbonate and 5.2% monoethylene glycol, condensed and recycled into the phase separation.

4.1 kg/h of the diisobutyl carbonate-rich phase from the phase separator are passed into the feed of the column for removal of the monoethylene glycol from the ethylene carbonate.

The monoethylene glycol-rich phase from the phase separator is purified by distillation at a mass flow rate of 0.76 kg/h in a column at a top pressure of 130 mbar and a bottom temperature of 139° C. This gives 0.72 kg/h of a liquid bottom stream with the composition of 99.99% monoethylene glycol and 60 ppm of ethylene carbonate. At the top of the column, 0.05 kg/h of a vapor stream are withdrawn with the composition of 23.8% diisobutyl carbonate, 66% isobutanol and 10.2% monoethylene glycol, condensed and recycled into the phase separation.

### Example 3

2.45 kg/h of ethylene carbonate are reacted with 5 kg/h of 1-propanol (molar ratio 1-propanol to ethylene carbonate 3:1) at 140° C. in the presence of 0.5 mol % (based on ethylene carbonate) of sodium isobutoxide as a catalyst in a tubular reactor to give 2.1 kg/h of dipropyl carbonate and 0.89 kg/h of monoethylene glycol.

The reaction output is passed into a dividing wall column which is operated at a top pressure of 150 mbar. The distillation is conducted continuously at a bottom temperature of 152° C. and a reflux ratio of 0.76 g/g.

At the bottom of the dividing wall column 1.89 kg/h of a liquid bottom product are obtained with a composition of 62.4% ethylene carbonate and 37.6% monoethylene glycol.

At the top of the dividing wall column, 5.8 kg/h of a vapor stream are obtained with the composition of 99.9% 1-propanol and 0.1% dipropyl carbonate, which is condensed and recycled into the reaction, or into the column as reflux.

In the side draw of the dividing wall column, 2.28 kg/h of a liquid biphasic stream are withdrawn, which comprises a composition of 92% dipropyl carbonate and 8% monoethylene glycol. This stream is fed to a phase separator. The phase separation into a dipropyl carbonate-rich phase with 98.2% dipropyl carbonate and a monoethylene glycol-rich phase with 97% monoethylene glycol is conducted at 40° C.

3.36 kg/h of the dipropyl carbonate-rich phase from the phase separator are conducted into a column and distilled at a bottom temperature of 127° C. and a top pressure of 300 mbar. 2.1 kg/h of liquid bottom product are obtained with a composition of 99.9% dipropyl carbonate and 100 ppm of monoethylene glycol. At the top of the column, 1.3 kg/h of vapor stream are withdrawn with a composition of 0.1% 1-propanol, 4.4% monoethylene glycol and 95.5% dipropyl carbonate, condensed and recycled into the phase separation.

14 kg/h of the dipropyl carbonate-rich phase from the phase separator are conducted into the upper part of a column for separation of the ethylene carbonate-monoethylene glycol mixture which is obtained at the bottom of the dividing wall column.

The column is operated at a top pressure of 70 mbar and a bottom temperature of 159° C. 1.2 kg/h of a liquid bottom stream are obtained with the composition of 99.9% ethylene carbonate and 0.1% catalyst, and also higher ethylene glycols, which is recycled into the reactor. A portion of the bottom stream is discharged to remove higher ethylene glycols from the system.

At the top of the column, 15.8 kg/h of a vapor stream are obtained with a composition of 93.5% dipropyl carbonate, 6.4% monoethylene glycol, 800 ppm of ethylene carbonate and 440 ppm of 1-propanol, which is condensed and recycled into the phase separation, or into the column as reflux.

The monoethylene glycol-rich phase from the phase separator is purified by distillation at a mass flow rate of 0.92 kg/h in a column at a top pressure of 100 mbar and a bottom temperature of 161° C. At the bottom, 0.89 kg/h of monoethylene glycol is obtained with a composition of 99.9% and 0.1% ethylene carbonate. At the top of the column, 0.03 kg/h of a vapor stream are obtained with a composition of 89.6% dipropyl carbonate, 7.2% monoethylene glycol, and 3.2% 1-propanol, which is condensed and recycled into the phase separation.

### LIST OF REFERENCE NUMERALS

1 Dividing wall column
2 Side feed
5 Stream comprising dialkyl carbonate and alkylene glycol as heteroazeotrope
7 Top stream comprising alcohol
9 Homoazeotrope-forming bottom stream comprising alkylene carbonate and alkylene glycol
11 Second distillation stage
13 Bottom stream comprising alkylene carbonate
15 Top stream comprising alkylene glycol and dialkyl carbonate
17 Side feed
19 Reactor
21 First feed
23 Second feed
25 Apparatus for phase separation
27 First crude product stream comprising essentially dialkyl carbonate
29 Second crude product stream comprising essentially alkylene glycol
31 Distillation column
33 Product stream comprising dialkyl carbonate
35 Distillation column
37 Product stream comprising alkylene glycol
39 First distillation column
41 Second distillation column

1. A process for obtaining a dialkyl carbonate and an alkylene glycol from a stream comprising dialkyl carbonate, alkylene carbonate, alkylene glycol and alcohol, comprising the following steps:
(a) distillatively removing a stream (5) comprising dialkyl carbonate and alkylene glycol as a heteroazeotrope from the stream comprising dialkyl carbonate, alkylene carbonate, alkylene glycol and alcohol in a first distillation stage (1),
(b) separating the stream (5) comprising dialkyl carbonate and alkylene glycol as a heteroazeotrope into a first crude product stream (27) comprising essentially dialkyl carbonate and a second crude product stream (29) comprising essentially alkylene glycol in an apparatus for phase separation (25).

2. The process according to claim 1, wherein the first distillation stage comprises a reactive distillation to which alkylene carbonate and alcohol are added as reactants and are converted in an equilibrium reaction to dialkyl carbonate and alkylene glycol, giving the stream comprising dialkyl carbonate, alkylene carbonate, alkylene glycol and alcohol.

3. The process according to claim 1, wherein the stream comprising dialkyl carbonate, alkylene carbonate, alkylene glycol and alcohol is supplied to the first distillation stage (1).

4. The process according to claim 1, wherein the first distillation stage (1) comprises a dividing wall column (1) in which the stream comprising dialkyl carbonate, alkylene carbonate, alkylene glycol and alcohol is separated into an alcohol-comprising top stream (7), a homoazeotrope-forming stream comprising alkylene carbonate and alkylene glycol as bottom stream (9) and the stream (5) which comprises dialkyl carbonate and alkylene glycol as a heteroazeotrope and is drawn off as a side stream, or wherein the first distillation stage comprises two distillation columns, the stream comprising dialkyl carbonate, alkylene carbonate, alkylene glycol and alcohol being separated in a first distillation column into a top stream comprising dialkyl carbonate, alkylene glycol and alcohol, and a homoazeotrope-forming bottom stream comprising alkylene carbonate and alkylene glycol, and the top stream comprising dialkyl carbonate, alkylene glycol and alcohol from the first distillation column being separated in a second distillation column into an alcohol-comprising top stream and the stream comprising dialkyl carbonate and alkylene glycol as a heteroazeotrope as the bottom stream.

5. The process according to claim 4, wherein the homoazeotrope-forming stream (9) comprising alkylene carbonate and alkylene glycol and a portion of the first crude product stream comprising essentially dialkyl carbonate are supplied as an entraining agent to a second distillation stage (11), and a bottom stream (13) comprising alkylene carbonate and a top stream (15) comprising dialkyl carbonate and alkylene glycol are withdrawn from the second distillation stage (11).

6. The process according to claim 1, wherein the first distillation stage comprises a dividing wall column in which the stream comprising dialkyl carbonate, alkylene carbonate, alkylene glycol and alcohol is separated into a bottom stream comprising alkylene carbonate, a top stream comprising alcohol, and the stream which comprises dialkyl carbonate and alkylene glycol as a heteroazeotrope and is drawn off as a side stream, or wherein the first distillation stage comprises a first distillation column (39) in which the stream comprising dialkyl carbonate, alkylene carbonate, alkylene glycol and alcohol is separated into a bottom stream (13) comprising alkylene carbonate and a top stream comprising dialkyl carbonate, alcohol and alkylene glycol, and a second distillation column (41) in which the top stream comprising dialkyl carbonate, alcohol and alkylene glycol from the first distillation column (39) is separated into an alcohol-comprising top stream (7) and the stream (5) comprising dialkyl carbonate and alkylene glycol as a heteroazeotrope as the bottom stream, and a portion of the first crude product stream comprising dialkyl carbonate is additionally added to the first distillation stage as an entraining agent.

7. The process according to claim 6, wherein the portion of the first crude product stream comprising dialkyl carbonate is added to the feed to the first distillation stage.

8. The process according to claim 6, wherein the portion of the first crude product stream comprising dialkyl carbonate is added on the feed side of the dividing wall column or to the first distillation column.

9. The process according to claim 1, wherein the first crude product stream (27) comprising essentially dialkyl carbonate is supplied to a distillative operation (31) in which alkylene glycol still present in the first crude product stream (27) comprising essentially dialkyl carbonate is removed therefrom to obtain a product stream (33) comprising dialkyl carbonate.

10. The process according to claim 9, wherein the alkylene glycol removed from the first crude product stream (27) comprising essentially dialkyl carbonate is supplied to the apparatus for phase separation (25) in step (b).

11. The process according to claim 1, wherein the second crude product stream (29) comprising essentially alkylene glycol is sent to a distillative operation (35) in which dialkyl carbonate still present in the crude product stream (29) comprising essentially alkylene glycol is removed therefrom to obtain a product stream (37) comprising alkylene glycol.

12. The process according to claim 11, wherein the dialkyl carbonate removed from the second crude product stream (29) comprising essentially alkylene glycol is supplied to the apparatus for phase separation (25) in step (b).

13. The process according to claim 1, wherein the alcohol is n-propanol, n-butanol, isobutanol, n-pentanol, n-hexanol, 2-methyl-1-butanol, 3-methyl-1-butanol or 2-methoxy-1-ethanol.

14. The process according to claim 1, wherein the alkylene carbonate is ethylene carbonate or propylene carbonate.
